(19) United States
(12) Patent Application Publication
(10) Pub. No.: US 2015/0335709 A1
(43) Pub. Date: Nov. 26, 2015

(54) Colostrum Composition Enriched in Anti-Endotoxin Antibodies

(71) Applicants: Alan Cross, Chevy Chase, MD (US); Zeil Rosenberg, Closter, NJ (US)

(72) Inventors: Alan Cross, Chevy Chase, MD (US); Zeil Rosenberg, Closter, NJ (US)

(73) Assignee: University of Maryland, Baltimore, Baltimore, MD (US)

(21) Appl. No.: 14/815,553

(22) Filed: Jul. 31, 2015

Related U.S. Application Data
(63) Continuation-in-part of application No. PCT/US2014/014095, filed on Jan. 31, 2014.
(60) Provisional application No. 61/758,954, filed on Jan. 31, 2013.

(51) Int. Cl. A61K 38/17; A61K 39/40; C07K 16/12; A61J 1/03

(52) U.S. Cl. CPC A61K 38/1709; A61J 1/035; A61K 39/40; C07K 16/1203

(57) ABSTRACT

The present invention provides a colostrum formulation enriched in broadly cross-reactive anti-endotoxin antibodies. Further provided is a method for reducing endotoxemia and blocking the onset of sepsis in patients comprising administering to the patients a colostrum formulation enriched with anti-endotoxin antibodies. Further provided is a method for treating an individual for hemolytic uremic syndrome or of protecting an individual against hemolytic uremic syndrome, comprising the step of administering to the patient an effective amount of the composition of the present invention.

### CROSS-REFERENCE TO RELATED APPLICATIONS

This application is a continuation-in-part under 35 U.S.C. §120 of pending international application PCT/US2014/014095, filed Jan. 31, 2014, which claims benefit of priority under 35 U.S.C. §119(e) of provisional application U.S. Ser. No. 61/758,954, filed Jan. 31, 2013, now abandoned, the entirety of both of which is hereby incorporated by reference.

### BACKGROUND OF THE INVENTION

The present invention generally relates to the field of immunology and infectious diseases. More specifically, the present invention relates to a colostrum composition enriched in anti-endotoxin antibodies and uses thereof.

### DESCRIPTION OF THE RELATED ART

Human colostrum, the first milk of mothers, is enriched in nutrients and non-specific immune factors that provide passive immunity until newborn immunity is established. It is widely recognized that colostrum and continued breast-feeding provide such health benefits that the U.S. Surgeon General has recommended breast-feeding guidelines to encourage more widespread breast feeding. As is the case for human colostrum, bovine colostrum (BC) is rich in immunoglobulins, antibacterial peptides, lactoferrin, cytokines and nutrients. Bovine IgG1, the main milk antibody, survives transit through the gut and may replace the need for secretory IgA. Bovine colostrum is widely considered safe and efficacious in treating diarrheal infection in humans, e.g., rotavirus, ETEC and enterohemorrhagic E. coli, including children. Antibodies in colostrum remain active in the intestinal tract. Surprisingly, oral bovine colostrum decreases the severity of viral upper respiratory tract infections in humans and influenza-immune bovine colostrum protects mice from experimental influenza, suggesting a potential benefit in respiratory infections as well.

There is increasing evidence that increases in gut permeability leading to Gram-negative bacterial endotoxemia plays a critical role in the morbidity/mortality associated with HIV infection, sepsis and malnutrition. Many conditions, including HIV infection, coronary bypass surgery and malnutrition, may impair gut barrier function leading to endotoxemia. In coronary bypass surgery, endotoxemia is associated with increased morbidity. In these patients, bovine colostrum reduced LPS translocation and systemic inflammation, a likely result of impaired gut barrier integrity. As bovine colostrum dramatically reduced stool frequency, increased body weight and CD4 counts in HIV-infected patients, hyperimmune bovine colostrum may be effective in HIV infection as well. During malnutrition, endotoxemia impairs immune cell function leading to recurrent infections, accelerates the development of AIDS in HIV-infected malnourished children and impedes growth (1-2). Thus, oral bovine colostrum enriched in anti-endotoxin antibodies may improve nutrition, immune and gut function and reduce infections during periods of impaired gut barrier integrity.

The prior art is deficient in colostrum compositions or formulations enriched with broadly cross-reactive anti-endotoxin antibodies and uses thereof. The present invention fulfills this longstanding need and desire in the art.

### SUMMARY OF THE INVENTION

The present invention is directed to a composition or formulation comprising colostrum enriched in broadly reactive anti-endotoxin antibodies.

The present invention is directed to a composition or formulation comprising colostrum enriched in anti-endotoxin antibodies.

The present invention is further directed to a method of reducing endotoxemia or blocking the onset of sepsis in patients comprising administering to patients a pharmaceutical composition or formulation comprising a colostrum formulation enriched with broadly reactive anti-endotoxin antibodies.

The present invention is further directed to a method of reducing endotoxemia or blocking the onset of sepsis in patients comprising administering to a patient a pharmaceutical composition or formulation comprising a colostrum formulation enriched with anti-endotoxin antibodies.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1D show serum IgG immune response of cows to J5dLPS/OMP vaccine. FIG. 1A: Cows in Cohort 1 were immunized with 3 doses of vaccine with Freund's Incomplete Adjuvant (FICA). Serum was collected before the first immunization (pre) and following each immunization just before the next immunization. A post-delivery booster dose of vaccine was administered after delivery (Post 4). Serum was also collected 7 days after the final (4^{th}) dose of vaccine. Data are expressed in Optical Density Units (ODU). FIG. 1B: Cows in Cohort 1 were immunized with 3 doses of vaccine with FICA. Serum was collected before the first immunization (pre) and following each immunization just before the next immunization. A post-delivery booster dose of vaccine was administered after delivery (Post 4). Serum was also collected 7 days after the final (4^{th}) dose of vaccine. Data are expressed in fold-increase of post-immune sera compared to pre-immune anti-J5 LPS levels. FIG. 1C: Cows in Cohort 2 were immunized with 3 doses of vaccine with the adjuvant Emulsigen-D®. Serum was collected before the first immunization (pre) and following each immunization just before the next immunization. Data are expressed in Optical Density Units (ODU). FIG. 1D: Cows in Cohort 2 were immunized with 3 doses of vaccine with Emulsigen-D®. Serum was collected before the first immunization (pre) and following each immunization just before the next immunization. Data are expressed in fold-increase of post-immune sera compared to pre-immune anti-J5 LPS levels.

FIGS. 2A-2F show class-specific serum immunoglobulin responses to J5dLPS/OMP immunization in serum samples obtained 3 weeks after third immunization of Cohort 1 and Cohort 2. Serum IgG, IgM and IgA antibody levels were measured by ELISA in samples obtained after the third immunization. Antibody levels are expressed in ODU. FIG. 2A: IgG antibody responses to J5dLPS/OMP immunization in serum samples obtained 3 weeks after third immunization of Cohort 1. FIG. 2B: IgM antibody responses to J5dLPS/OMP immunization in serum samples obtained 3 weeks after third immunization of Cohort 1. FIG. 2C: IgA antibody responses to J5dLPS/OMP immunization in serum samples obtained 3 weeks after third immunization of Cohort 1. FIG. 2D: IgG antibody responses to J5dLPS/OMP immunization in serum samples obtained 3 weeks after third immunization of Cohort 2. FIG. 2E: IgM antibody responses to J5dLPS/OMP immunization in serum samples obtained 3 weeks after third immunization of Cohort 2. FIG. 2F: IgA antibody responses to J5dLPS/OMP immunization in serum samples obtained 3 weeks after third immunization of Cohort 2.

FIGS. 3A-3C shows the comparison of immunoglobulins G, A and M in pre-immunization milk obtained at dry-off to colostrum obtained within first day of delivery. Milk was obtained at dry-off and colostrum within first day of delivery for Cohort 1 and isotype-specific antibody to J5 LPS determined by ELISA. The anti-J5 LPS IgG, IgA and IgM antibody levels were determined by ELISA and expressed as ODU. FIG. 3A: Comparison of IgG in pre-immunization milk to IgG in colostrum. FIG. 3B: Comparison of IgA in pre-immunization milk to IgA in colostrum. FIG. 3C: Comparison of IgM in pre-immunization milk to IgM in colostrum.

FIGS. 4A-4B show the levels of anti-J5 LPS antibodies in the serum and colostrum following immunization with the J5dLPS/OMP vaccine. The levels of colostral IgA and IgG as well as serum IgG levels are expressed as ODU. FIG. 4A: The level of IgG antibody in the serum obtained 2 weeks after the third immunization was compared to the anti-J5 LPS antibody levels in the colostrum obtained within one day of delivery in cows in Cohort 1. FIG. 4B: The level of IgG antibody in the serum obtained 2 weeks after the third immunization was compared to the anti-J5 LPS antibody levels in the colostrum obtained within one day of delivery in cows in Cohort 2.

FIG. 5 shows the effects of immunization of mice with J5 Bacterin® vs. J5dLPS/OMP vaccine. After obtaining pre-immunization sera from retro-orbital bleeds, outbred CD-1 mice were immunized intraperitoneally three times with either J5 Bacterin® or J5dLPS/OMP vaccine (200 μl). Sera obtained 14 days after the third bleed were assayed for antibody levels to J5 dLPS. The serum levels of anti-J5 LPS IgG were greater in mice immunized with the J5dLPS/OMP vaccine than with the J5 Bacterin® vaccine (p<0.02 by ANOVA and Student t test)

FIG. 6 shows that endotoxemia is reduced in neutropenic rats pretreated with J5 LPS/OMP hyperimmune colostrum compared to an equal volume of cow's milk. Rats were rendered neutropenic with cyclophosphamide and treated with moxafloxacin to overcome colonization resistance before oral infection with P. aeruginosa (10⁶ CFU on days 0, 2, and 4). Rats were administered by orogastric feeding (2 mls/day on days 3 and 5) colostrum from either cow #292 (n=8) or 423 (n=8) or an equal volume of milk or saline as negative controls (n=16). The circulating endotoxin levels were measured from sera sterilely obtained at day 5 following infection and measured by turbidometric Limulus Amebocyte Lysate assay. The circulating endotoxin levels at day 5 after infection (onset of fever) are represented as the median values with 95% confidence intervals and analyzed by the Kruskal-Wallis method for non-parametric data in multiple groups. Circulating endotoxin levels decreased following administration of bovine colostrum (p=0.027)

FIGS. 7A-7F show serum IgG1 and IgG2 subclass responses to immunization with J5dLPS/OMP vaccine. FIG. 7A: Serum IgG1 antibody responses in ODU were measured before and following each immunization for Cohort 1. FIG. 7B: Serum IgG2 antibody responses in ODU were measured before and following each immunization for Cohort 1. FIG. 7C: Serum IgG1/IgG2 ratios before and following each immunization for Cohort 1. FIG. 7D: Serum IgG1 antibody responses in ODU were measured before and following each immunization for Cohort 2. FIG. 7E: Serum IgG2 antibody responses in ODU were measured before and following each immunization for Cohort 2. FIG. 7F: Serum IgG1/IgG2 ratios before and following each immunization for Cohort 2.

FIG. 8A-8B show comparison of serum anti-J5 LPS IgG levels of cows immunized with J5dLPS/OP vaccine to anti-J5 LPS IgG levels of a cow hyperimmunized with J5 Bacterin® vaccine. Cows from Cohorts 1 and 2 were immunized with the J5dLPS/OMP vaccine and sera were obtained before initial (pre) immunization and after the third immunization (post3). Plasma also was obtained from a cow who was immunized on multiple occasions with commercially-obtained Bacterin J5® and SP Toxoid® vaccines (hyperimmune plasma). FIG. 8A: Serum IgG antibody levels measured by ELISA with J5 LPS as the capture antigens on the plate. Data are expressed as ODU. FIG. 8B: Serum IgG antibody levels measured by ELISA with J5 Bacterin® as the capture antigen on the plate. Data are expressed as ODU.

FIGS. 9A-9B show that J5dLPSOMP immunization prevents weight loss and improves survival of mice given Shiga-like toxin 2 and LPS. FIG. 9A: C57BL/6 male mice were immunized with the J5dLPS/OMP vaccine on days 0, 14 and 28. Control mice received an equal volume of PBS. At 14 days after the last dose of vaccine, mice were given Shiga toxin 2 (Stx2) at 5 ng/20 g body weight either alone (S(5)), or with LPS 1 μg/20 g body weight (L(1)). They were then followed for weight change. FIG. 9B: C57BL/6 male mice were immunized with the J5dLPS/OMP vaccine on days 0, 14 and 28. Control mice received an equal volume of PBS. At 14 days after the last dose of vaccine, mice were given Shiga toxin 2 (Stx2) at 5 ng/20 g body weight either alone (S(5)), or with LPS 1 μg/20 g body weight (L(1)). The animals were then followed for survival.

FIG. 10 shows that J5dLPS/OMP immunization reduces circulating endotoxin in mice administered Stx2/LPS. C57BL/6 mice were immunized with the J5dLPS/OMP vaccine (J5_CPG+S(5)L(6)) and a control group received an equal volume of PBS (PBS_S(5)L(6)). Fourteen days after immunization mice were challenged with Stx2 at 5 ng/20 g body weight (S(6)) and LPS at 6 μg/20 g body weight (L(6)). Plasma was obtained at 24 hr after challenge and endotoxin levels were measured with a quantitative Limulus Amebacyte Lysate assay.

FIG. 11 shows that the comparison of Kaplan-Meier Survival curves for an animal model of sepsis. On days 3-8 rats were treated orally with 2 ml of phosphate buffered saline, non-immune bovine colostrum or hyperimmune bovine colostrum from cows immunized with the J5/OMP vaccine. The animals are challenged orally with a lethal isolate of Pseudomonas aeruginosa. The survival rate for each treatment was recorded for 336 hours.

### DETAILED DESCRIPTION OF THE PRESENT INVENTION

Unless otherwise noted, technical terms are used according to conventional usage in the art.

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any method, compound, composition, or device described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “about” refers to a numeric value, including, for example, whole numbers, fractions, and percentages, whether or not explicitly indicated. The term “about” generally refers to a range of numerical values, e.g., +/−5-10% of the recited value, that one of ordinary skill in the art would consider equivalent to the recited value, e.g., having the same function or result. In some instances, the term “about” may include numerical values that are rounded to the nearest significant figure.

As used herein, “effective amount” means that amount or dosage which will bring about the desired result, i.e reduction of endotoxemia or blockage of blood infection. The selected dosage may be administered in one dose or in intermittent dosages. The compositions may be provided for humans or animals in liquid or powdered form to be mixed with any of a variety of raw, processed, cooked or uncooked foods. A dosage unit may be administered separately or together with other nutritional products such as vitamins and minerals.

Gut-derived translocation of gram negative bacterial lipopolysaccharide (LPS, or endotoxin) increasingly has been identified as a source of systemic inflammation that exacerbates chronic conditions such as HIV, cardiovascular and inflammatory bowel diseases and malnutrition. There is a tremendous need to identify alternative therapeutic strategies that are administered more easily and cost effectively compared to current treatment protocols. Colostrum is a unique product that arises from a distinct physiological and functional state of the mammary gland. The levels of antibodies are much higher in the colostrum than those observed using the conventional methods in the art. The oral administration of colostrum, such as bovine colostrum, reduces endotoxemia in patients undergoing coronary bypass graft surgery and improves gut function in patients infected with HIV. Patients with low levels of serum antibody to highly conserved epitopes in the core region of endotoxin, as measured by EndoCab® ELISA (Hycult Biotech), are more likely to have complications after coronary artery bypass graft surgery. Consequently, colostrum enriched in broadly reactive antibodies to LPS may ameliorate endotoxemia-related morbidities. The vaccine used in the present invention utilizes a detoxified lipopolysaccharide from a strain of E. coli complexed with an outer membrane protein (OMP) of a bacterium such as N. meningiditis with utility as a vaccine against a wide variety of enteric bacteria. This approach yields much higher levels of broadly reactive anti-endotoxin antibodies to the conserved epitopes in the colostrum as compared to current technology such as the Bacterin® vaccine which is comprised of whole, killed bacteria.

In one embodiment, the present invention provides a composition comprising colostrum enriched in broadly reactive anti-endotoxin antibody. Generally, the level of broadly reactive anti-endotoxin antibodies in the enriched colostrum composition is from about 10 mg/ml to about 500 mg/ml. The anti-endotoxin antibodies in the enriched colostrum composition have high levels of cross reactivity with gram negative bacteria. Representative gram negative bacteria to which the anti-endotoxin antibodies in the enriched colostrum composition may cross react with include, but are not limited to, Enterobacteriaceae, such as Salmonella spp., Yersinia spp., Klebsiella spp., Shigella spp., Serratia spp., Helicobacter spp., Vibrio spp., and Pseudomonas spp., for example, Pseudomonas aeruginosa. The colostrum of the present invention may be obtained from any suitable ungulate, but representative examples include, although are not limited to, bovine colostrum, including buffalo colostrum or bison colostrum, caprine colostrum, porcine colostrum, or equine colostrum as well as other sources such as camelid colostrum, including camel and llama colostrum.

In another embodiment, the present invention provides a pharmaceutical composition, comprising colostrum enriched in broadly reactive anti-endotoxin antibodies. Representative formulations for the administration of this pharmaceutical composition include but are not limited to liquids, tablets, powders contained in a capsule, blister pack or sachet.

In another embodiment, the present invention relates to a method for reducing endotoxemia in a patient or for blocking the onset of a blood infection of a patient, comprising the step of administering to the patient an effective amount of a pharmaceutical composition comprising of a colostrum formulation enriched with anti-endotoxin antibodies. In a preferred embodiment, the pharmaceutical composition comprising a colostrum formulation enriched with broadly reactive anti-endotoxin antibodies is formulated as an oral dose formulation. Representative oral dose formulations for the administration of the pharmaceutical composition comprising of a colostrum formulation enriched with anti-endotoxin antibodies include but are not limited to liquids, tablets, or powders contained in a capsule, blister packs or sachet. The pharmaceutical composition comprising of a colostrum formulation enriched with anti-endotoxin antibodies is administered to a patient in a dose of preferably from about 3 grams to about 50 grams.

In preparing the compositions or formulations in oral dosage form, any of the usual pharmaceutical carriers may be employed. For oral liquid preparations, e.g., suspensions, elixirs, and solutions, and for carriers containing water, oils, alcohols, flavoring agents, preservatives, coloring agents and the like may be used. Carriers such as starches, sugars, diluents, granulating agents, lubricants, binders, disintegrating agents, and the like may be used to prepare oral solids, e.g., powders, gelatin capsules, pills, and tablets. Lozenges, chewable tablets and controlled release forms may also be used. If desired, tablets may be sugar coated or enteric coated by standard techniques. Examples of additional inactive components which provide for easier oral administration include but are not limited to beeswax, lecithin, gelatin, purified water, and glycerin. As stated above, oral dosage formulations of the compositions may be added to foods and consumed. Any conventional foodstuff may be appropriate as a carrier for the presently disclosed compositions.

The following example(s) are given for the purpose of illustrating various embodiments of the invention and are not meant to limit the present invention in any fashion.

### Example 1

### Cows

Two cohorts comprised of three and four cows respectively were selected based on the expectation to deliver within 3 months and were immunized in this study as shown in Table 1. Both were from organically-maintained herds in rural Pennsylvania.

TABLE 1

| | | | Previous | | | Last dose relative |
|---|---|---|---|---|---|---|
| | Breed | Age | Deliveries | Dose | | to time of calving |
| Cohort 1 | | | | | | |
| a. 317 | Jersey/ | 8 | 6 | 440 | mcg | Sep. 2, 2011 |
| | Holstein | | | | | 8 D |
| b. 292 | Jersey/ | 8 | 6 | 440 | mcg | Oct. 8, 2011 |
| | Holstein | | | | | 44 D |
| c. 423 | Jersey/ | 5 | 3 | 1,760 | mcg | Sep. 10, 2011 |
| | Holstein | | | | | 16 D |
| Cohort 2 | | | | | | |
| d. Melody | Holstein | 4 | 3 | 1,100 | mcg | Jan. 20, 2012 |
| | | | | | | 17 D |
| e. Cheryl | Holstein | 4 | 3 | 275 | mcg | Feb. 9, 2012 |
| | | | | | | 37 D |
| f. Cari | Holstein | 1 | 0 | 275 | mcg | Feb. 16, 2012 |
| | | | | | | 44 D |
| g. Marsha | Holstein | 2 | 0 | 1,100 | mcg | Jan. 14, 2012 |
| | | | | | | 11 D |

Cows in cohort 1 were immunized subcutaneously at alternating sites on Jun. 29, 2011 (Day 0), Jul. 14, 2011 (Day 16), Aug. 8, 2011 (Day 41) and Aug. 25, 2011 (Day 58) with indicated dose of vaccine in 4 ml+4 ml of Freund's Incomplete Adjuvant (FICA) (total 8 ml/dose). On Day 58, no FICA, but 2× dose, same volume. Each cow was vaccinated 1.5 yrs earlier with Scourgard 4KC® which has a coliform component, but at the time of J5dLPS/OMP immunization was at the tail end of any expected protection.

Cows in cohort 2 were immunized subcutaneously at alternating sites on Nov. 22, 2011 (Day 0), Dec. 9, 2011 (Day 18) and Jan. 3, 2012 (Day 43) and then Day 117 (post-delivery) with indicated dose of vaccine in 2.4 ml+0.6 ml of Emulsigen-D® (total 3.0 ml/dose). All were previously immunized with Bovishield® which contains BVD, BRSV, PI3, IBR and leptospira antigens, but none received any coliform-containing vaccine.

### Example 2

### Vaccine

Vaccine was made under non-GMP conditions as described by Bhattacharjee et al. (3). Briefly, E. coli J5 LPS was purchased from List Biologics and treated with NaOH. Group B meningococcal outer membrane protein (OMP) was obtained from Dr. Kenneth Eckels at the Bioproduction Pilot Facility of the Walter Reed Army Institute of Research. The dLPS and outer membrane protein were mixed in a ratio of 1:1.2 (w/w LPS/OMP). Each 4 ml vial of vaccine contained 1,760 mcg of dLPS (undiluted for cow #423) or 440 mcg/ml (1:4 dilution for cows #292 and 317).

### Example 3

### Bovine Immunization and Sample Collections

Two cohorts of cattle were immunized at alternating sites. Milk was obtained at dry-off from all four quadrants of each cow's udder as a pre-immunization control. For cohort 1, blood was obtained from the coccygeal vein for serum antibody determinations before each of four immunizations and 8 days after the fourth immunization. The vaccines were mixed 1:1 (vol/vol) with Freund's Incomplete Adjuvant and the 8 ml were given subcutaneously (SQ) initially at the left side of the neck. A second dose of vaccine was given 15 days later on the right side of the neck and a third dose of vaccine 35 days after the second dose on the right side of the neck. A fourth dose of vaccine was given 17 days later in the right thorax. For this final immunization the dose of vaccine was doubled and Freund's Incomplete Adjuvant was omitted (total volume=8 ml). Colostrum was obtained from each cow within a day of delivery and for 5 days thereafter (8-44 days after the last dose of vaccine). Blood was obtained from the caudal vein for serum antibody determinations just before the second immunization (i.e. 15 days). Blood was obtained 25 days after the second immunization and before administration of the 3 d dose. Blood was obtained 17 days after the 3 d dose of vaccine, just before the 4^{th} dose of vaccine, and a final bleed was performed 8 days later.

For Cohort 2, a veterinary adjuvant that is less reactogenic, Emulsigen-D®, was used instead of Freund's Incomplete Adjuvant because it cannot be used as an adjuvant in cows destined for human consumption. 2.4 ml of vaccine was mixed with 0.6 ml of Emulsigen-D (MVP Laboratories, Inc, Omaha, Nebr.) for a total volume of 3.0 ml. Blood was obtained before each of three immunizations and at day 7 post-final immunization. After obtaining milk and blood, the vaccine initially was given in the left neck subcutaneously. A second dose was administered in the right neck 17 days later, with a third dose of vaccine 25 days after the second dose in the left neck.

### Example 4

### Murine Immunizations

To compare the immunogenicity of the J5dLPS/OMP vaccine with the commercially-available Bacterin® vaccine (Poultry Health Labs, Division of PHL Associates, Inc. Davis, Calif.; now Zoetis J5), mice (ICR mice, Charles River) were immunized and their antibody responses were measured by a previously described ELISA assay (3). Antibody responses were also compared to those of a cow that was hyperimmunized with the J5 Bacterin® vaccine as well as another vaccine (Salmonella typhimurium Bacterin-Toxoid, Re-17 derived mutagenically (Endovac-Bovi R with Immune Plus R (IMMVAC, Inc. Columbia, Mo.)). Outbred mice (6-8 weeks old females) were immunized intraperitoneally at days 0, 14 and 28 in a volume of 200 ul. Mice received PBS, Bacterin (200 μl) or J5dLPS/OMP complex vaccine (22 mcg dLPS) and were bled 7 days after the third immunization.

### Example 5

### ELISA Assay

An ELISA assay was adapted to measure the antibody levels in the serum, milk and colostrum of cows (3). Briefly, after addition of poly-L-lysine to wells in a 96-well microtiter plate, J5 LPS was added as the coating antigen. Bovine samples were then added to the wells and followed by HRP-conjugated anti-bovine IgG, IgA and IgM secondary antibodies. Substrates TMB Peroxidase EIA Kit, BIO-RAD) were added to the wells and the reactions were stopped with 1 N sulfuric acid. The amount of antibody was expressed in optical density units (ODU) that was derived from the optical density reading from the linear portion of the ELISA curve (usually at an OD ˜0.2) multiplied by the dilution at which that reading was obtained.

### Example 6

### Reduction of Endotoxemia in Neutropenic Rat Model of “Leaky Gut”

Female albino Sprague-Dawley rats (150-200 gram specific pathogen-free; Charles River Laboratories) were housed in environmentally isolated cages and maintained in a constant ambient temperature and humidity in a twelve hour day/night cycling. Animals were provided with an ad-libitum supply of commercial rodent chow and distilled water. The animals were allowed to adjust to the laboratory facilities for at least 7 days before undergoing any experimental procedures. Rats were rendered neutropenic by treatment with cyclophosphamide and colonization resistance overcome with antibiotics using a standard protocol (4). The treatment group (N=16) received the hyperimmune colostrum from either cow #292 or cow #423 by orogastric feeding (2 ml/day) on days 3 and 5 while the control group (n=16) were given either saline or cow's milk as negative controls. Following oral challenge with P. aeruginosa (10⁶ CFU on D0, 2 and 4), rats typically develop fever and endotoxemia starting at day 5 after infection. Endotoxin levels were measured on day 5 from heat-treated plasma samples using the quantitative Limulus Amebocyte Lysate assay using standard methods (5).

### Example 7

### Reactogenicity

Each of the cows tolerated the immunization well. A cow in Cohort 1 had an indurated area over each of the immunization sites that persisted for the entire 6 week observation period. In contrast, in Cohort 2, Emulsigen-D did not result in any induration.

### Example 8

### Immunization with the J5dLPS/OMP Vaccine Induces a Serum Antibody Response

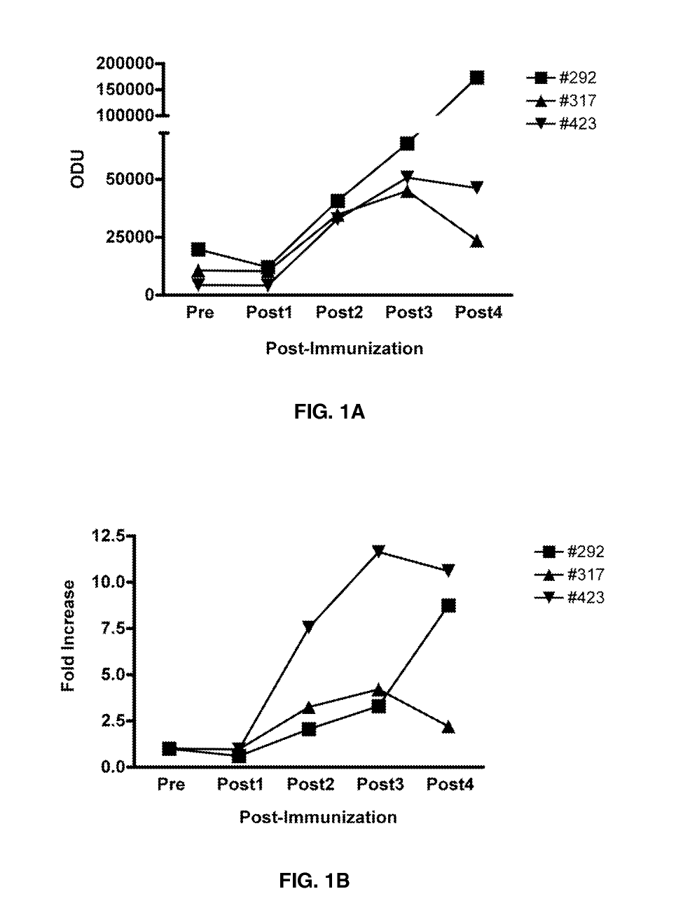

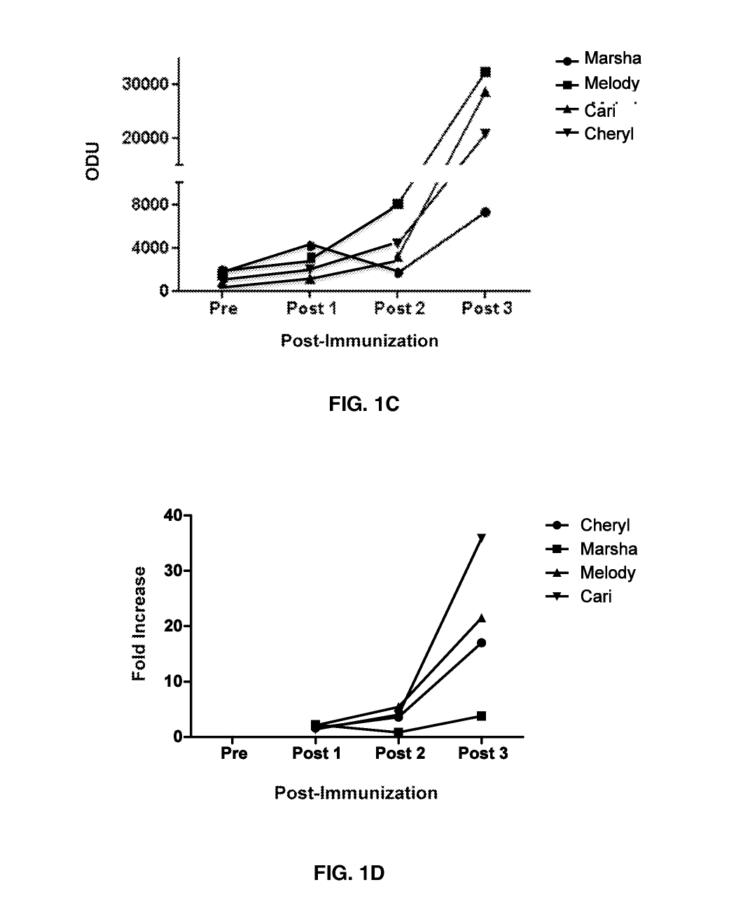

In the first cohort, after the first immunization there was no increase over baseline IgG levels, but following the second immunization at 4 weeks there was a 2.5-7.5-fold increase in IgG antibody levels over baseline (FIGS. 1A-1B). This increased slightly after the third immunization in two of the cows and markedly in the third. A fourth immunization in the absence of adjuvant at 8 weeks resulted in a further increase in IgG antibody level. (Note difference in ODU vs fold-increase for #292 and 423 which probably represents differences in baseline levels). There were less robust serum IgA and IgM antibody responses: the IgM increased by >4-fold in only one of the 3 cows, while peak serum IgA titers only doubled over baseline in all 3.

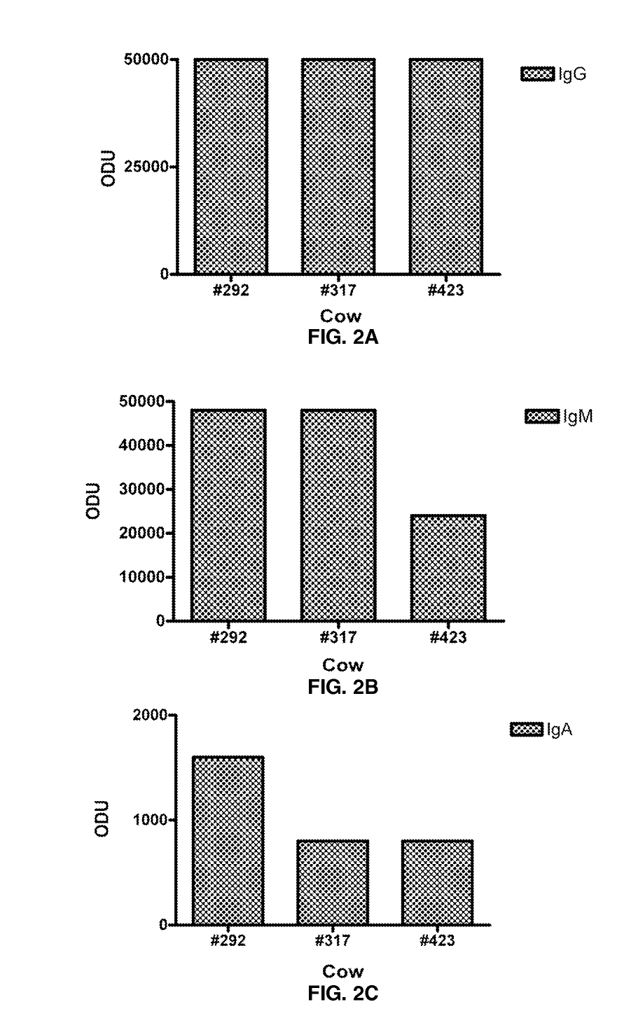

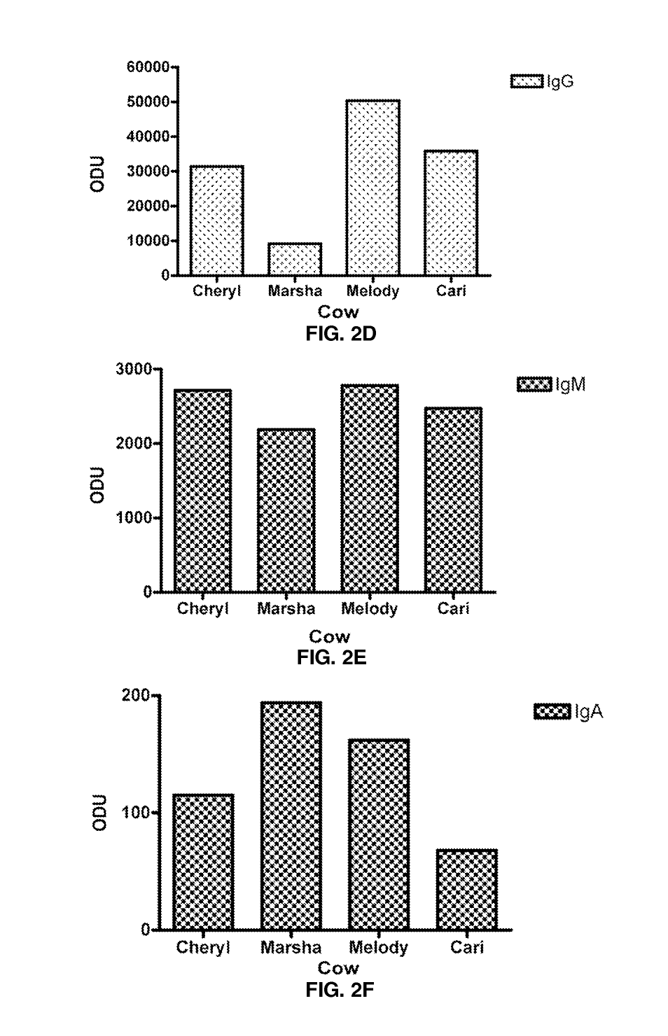

The cows in Cohort 2 were immunized at different anatomic sites with the vaccine in conjunction with the veterinary adjuvant, Emulsigen-D®, an oil-in-water adjuvant preparation. Robust serum antibody responses after the third immunization were observed in three of the four cows (FIGS. 1C-1D). The fourth, Marsha, while mounting a good IgG antibody response (7,500 ODU) was not as great as the other three. In two of the 4 cows (Marsha and Melody) there was a >2-fold increase in IgG over baseline after the first immunization and in 3 of the 4 cows there was a marked increase after the second immunization (except for Marsha). There was a highly significant increase in serum IgG after the 3 d immunization in three of the four cows. The serum IgM and IgA responses after the third dose of vaccine were not as robust as the IgG responses (FIGS. 2D-2F). Each of the four cows had similar serum IgM responses and, except for Cari, similar serum IgA responses. The serum IgM level increased 2-fold after the first immunization in two of the 4 cows, and then returned to baseline levels (data not shown). The serum IgA increased after the first immunization in only one (Melody) of the four cows.

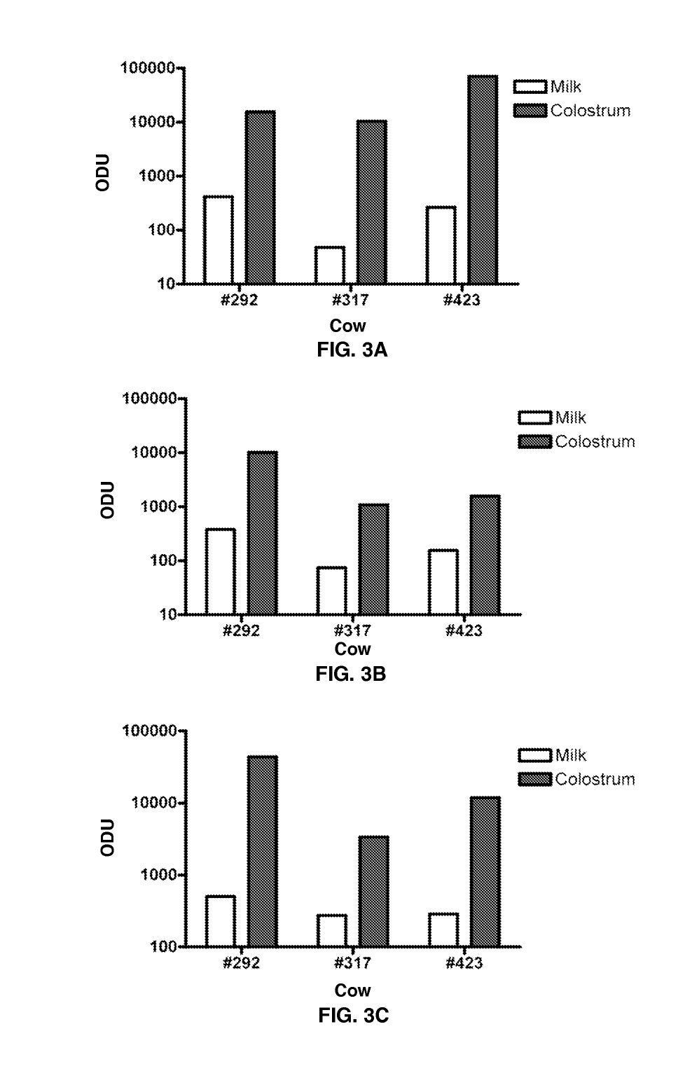

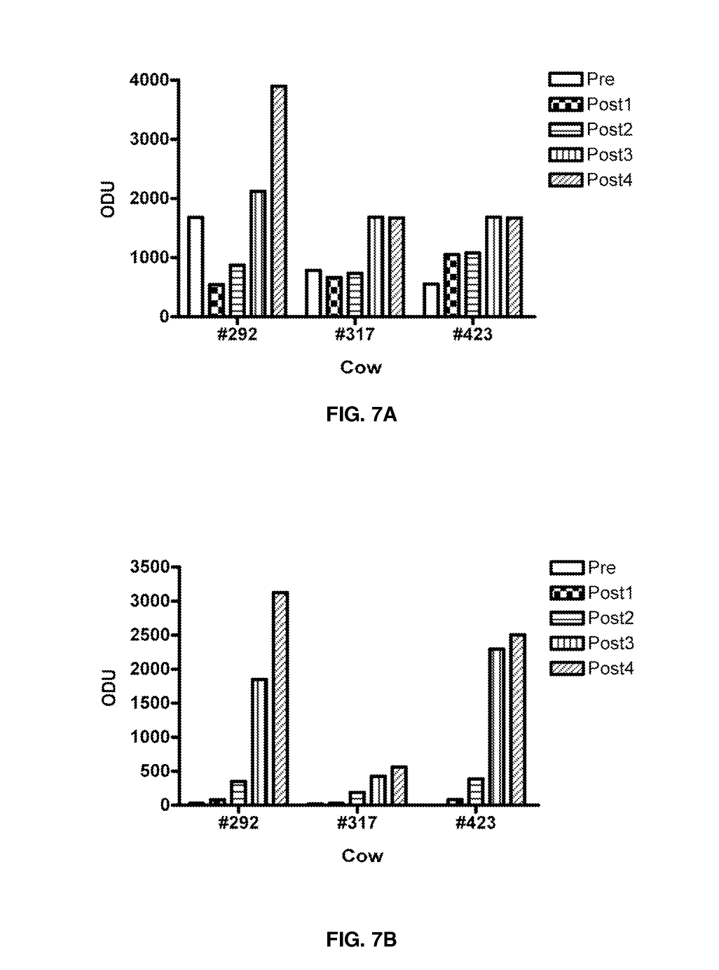

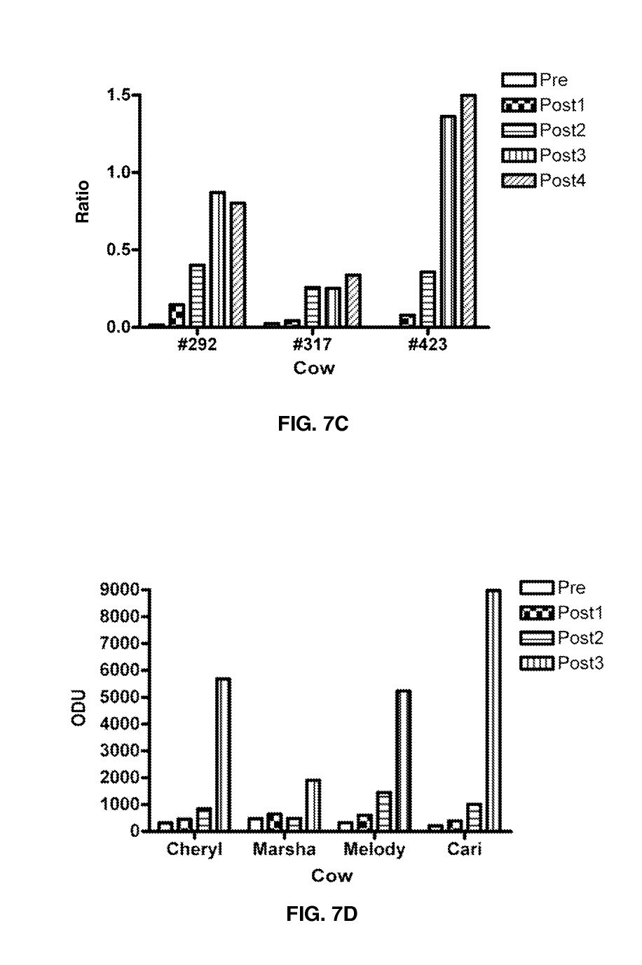

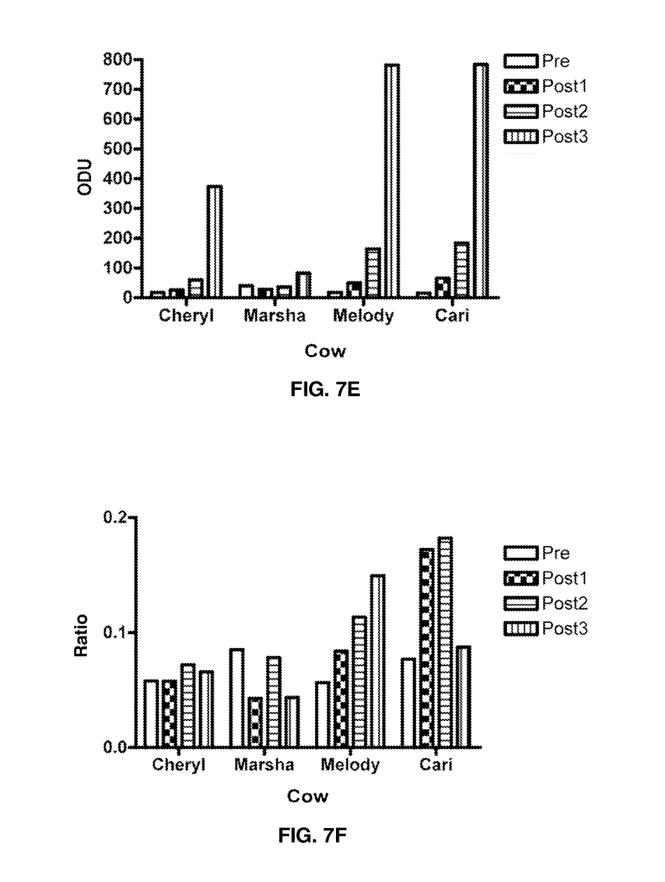

The IgG1 and IgG2 subtype serum antibody responses were also examined. In Cohort 1 there was a progressive increase in IgG2 subtype with each immunization (FIG. 3B), particularly after the third and fourth immunizations, and the IgG2/IgG1 ratio also increased progressively with each immunization (FIG. 7C). In Cohort 2, while the IgG2 subtype increased markedly over pre-immunization baseline in 3 of the 4 cows (FIG. 7E), the IgG1/IgG2 ratio increased for only 2 of the 4 cows (FIG. 7F), due in large part to the robust IgG1 responses as well (FIG. 7D).

### Example 9

### Colostral Anti-J5 LPS Antibody Response

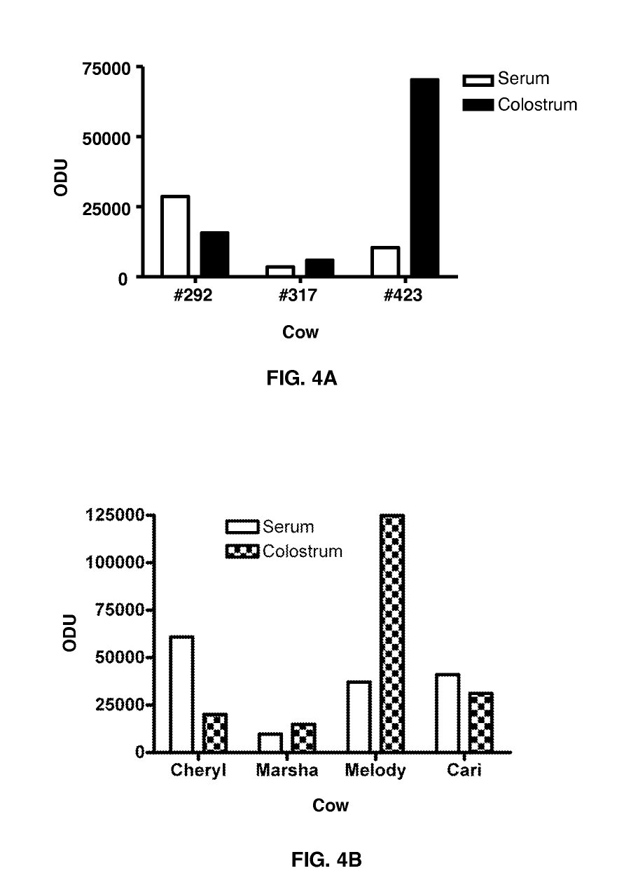

Before immunization, milk was obtained at dry-off from all four quadrants as a pre-immunization control. Colostrum from each of the immunized cows was obtained within a day of delivery and antibody response compared to levels in milk at dry-off. In Cohort 1 the anti-J5 LPS IgG antibody levels were increased in the colostrum compared to that in the milk (FIG. 3A). There was a 12 to nearly 370 fold increase in anti-J5 LPS IgG compared to levels in milk, while there was a 9-22 fold increase in colostral IgA and 9 to 95 fold increase in colostral IgM (FIGS. 3B-3C). Of interest, the cow (#292) that had the greatest level of IgA and IgM antibody did not have the greatest colostral IgG response (FIG. 4A). The colostral anti-J5 LPS IgG was increased above the serum level in one (#423) of the cows and lower than the serum IgG level in another (#292) (FIG. 4A). Table 2 shows the levels of anti-J5 LPS antibodies in the serum and colostrum following immunization of cohort 1 with J5dLPS/OMP vaccine. The levels of colostral IgA and IgG as well as serum IgG levels are expressed as ODU. The colostral IgG/serum IgG ratio was determined.

TABLE 2

| | |
|---|---|
| Colostrum | Serum |

| Cow | IgA | IgG | IgG | IgG Ratio |
|---|---|---|---|---|
| #292 | 10108 | 15686 | 28748 | 0.55 |
| #317 | 1090 | 10444 | 3597 | 2.90 |
| #423 | 1581 | 70320 | 10444 | 6.73 |

In Cohort 2, each of the 4 cows had robust serum IgG antibody responses, each exceeding 10,000 ODU, with Marsha being lower than the other three. The J5 LPS IgG responses in the colostrum did not appear to follow any pattern with regard to the IgG level in the serum. The cow with the greatest colostral IgG level (Melody) had a level of serum IgG antibody similar to Cari, but had nearly a 10-fold greater colostral antibody level. Cows with similar levels of colostral IgG (Cheryl and Marsha) had nearly a 6-fold difference in serum IgG. Each of the cows expressed J5-LPS-specific IgA in the colostrum (FIG. 4B). Table 3 shows the levels of anti-J5 LPS antibodies in the serum and colostrum following immunization of cohort 2 with J5dLPS/OMP vaccine. The levels of colostral IgA and IgG as well as serum IgG levels are expressed as ODU. The colostral IgG/serum IgG ratio was determined.

TABLE 3

| | |
|---|---|
| Colostrum | Serum |

| Cow | IgA | IgG | IgG | IgG Ratio |
|---|---|---|---|---|
| Cheryl | 2595 | 13320 | 60840 | 0.22 |
| Marsha | 2250 | 10800 | 9725 | 1.11 |
| Melody | 3880 | 162300 | 37102 | 4.37 |
| Cari | 2100 | 18940 | 41084 | 0.46 |

### Example 10

### Comparison of Immunogenicity of Bacterin to that of J5dLPS/OMP in Mice

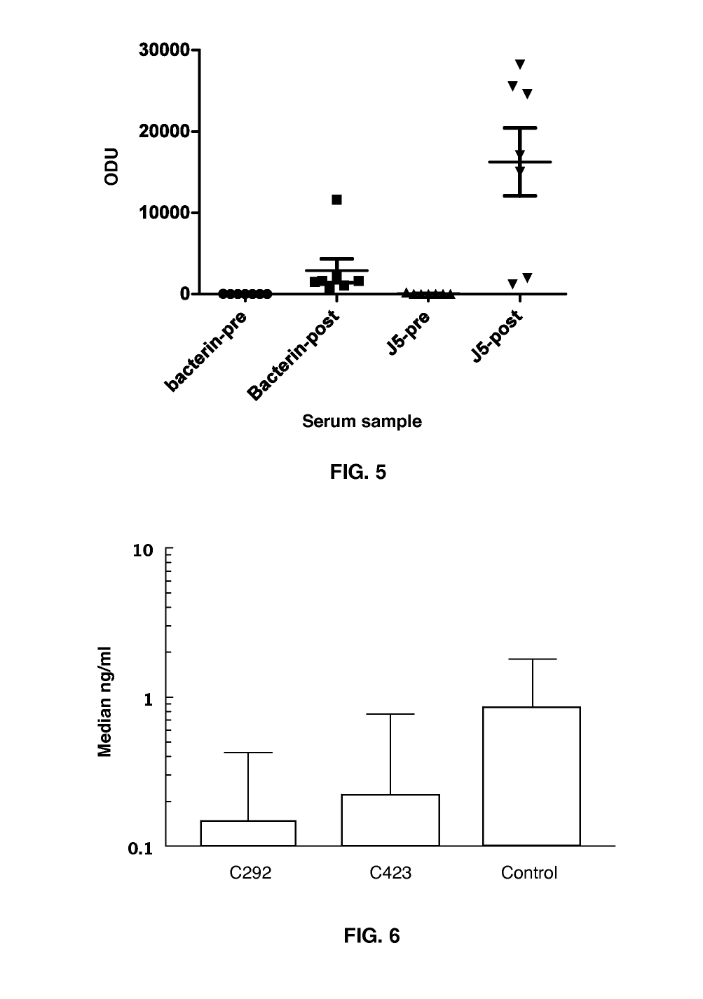

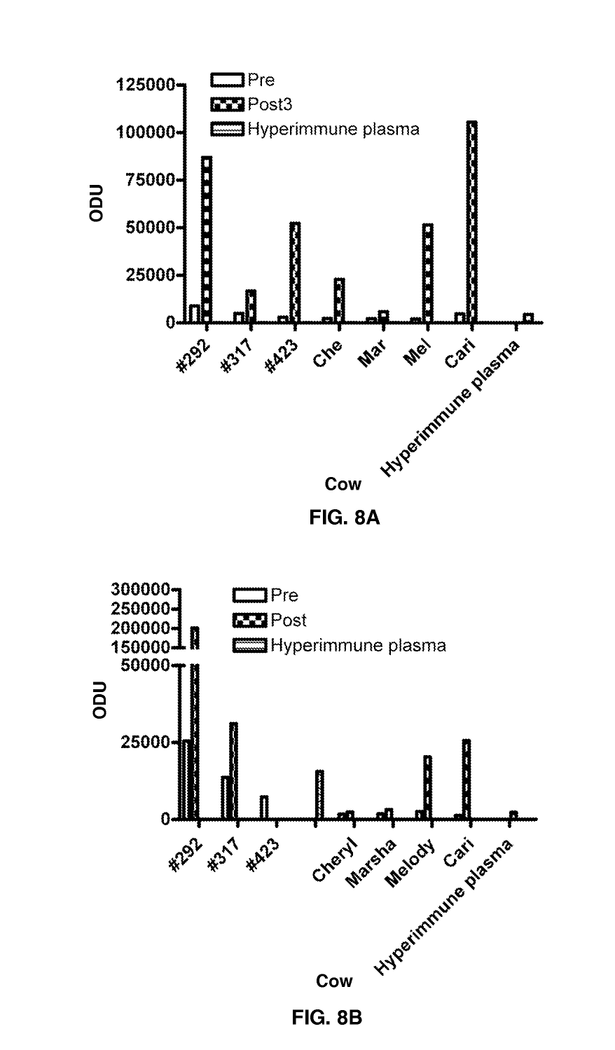

The J5dLPS/OMP vaccine contains purified LPS prepared from the E. coli O111:H4 bacterial strain, with each human dose having 10 mcg of the LPS. In contrast, each recommended dose of J5 Bacterin® contains ˜10⁹ CFU/ml of the heat-killed whole bacteria (Manufacturer data sheet), which can be estimated to have ˜2.5 mcg of LPS (6). Assuming that the J5 LPS is the active immunogen in the J5 Bacterin®, the J5dLPS/OMP vaccine has about 40 times the LPS content per dose as the J5 Bacterin® (0.5 μg vs. 22 μg). To compare the ability of the two vaccines to induce an antibody response to J5 LPS, mice were immunized with 200 μl of either the vaccine (22 μg J5dLPS) or Bacterin® (˜2×10⁸ CFU) and the IgG antibody response to J5 LPS was measured by ELISA one week after the third dose. Immunization with J5 Bacterin® induced IgG antibody levels between 649 and 11,600 ODU (2,883±3,872) (FIG. 5). Administration of three doses of the J5 dLPS/OMP vaccine at the same 4 week intervals resulted in a 5-fold greater mean IgG antibody level (FIG. 5). Immunization with the J5dLPSOMP vaccine also induced robust levels of antibody against antigen (FIG. 8B).

### Example 11

### Functional Antibody Response

Administration of hyperimmune bovine colostrum reduced endotoxemia in an animal model of “leaky gut”, not unlike that found in clinical situations. Rats underwent cyclophosphamide treatment to induce neutropenia and antibiotics to reduce colonization resistance before being given P. aeruginosa by gavage. Typically, rats become systemically infected by day 5 as manifested by development of fever, bacteremia and endotoxemia. Rats were given either J5 LPS antibody-enriched bovine colostrum on days 3 and 5, or either milk or saline as controls, and circulating endotoxin levels were measured by Limulus Amebacyte Lysate assay. Rats that received bovine colostrum enriched in anti-J5 LPS had lower circulating endotoxin levels at the onset of fever than those that received control treatment (FIG. 6).

### Example 12

### Comparison of J5 LPS Serum IgG Responses to the J5dLPS/OMP Vaccine and to J5 Bacterin®

J5 Bacterin® is a licensed anti-endotoxin vaccine made from the same parent bacterial strain as the J5dLPS/OMP vaccine. Thus, the antibody responses of the J5dLPS/OMP vaccine was compared to that of J5 Bacterin® as well as to other anti-sepsis vaccines. A cow was hyperimmunized with J5 Bacterin® and SP toxoid vaccines and the hyperimmune plasma was harvested. Each of the seven cows had antibody levels to J5 LPS that greatly exceeded those in the hyperimmune plasma (FIG. 8A). Each of the cows immunized with the J5dLPS/OMP vaccine had serum IgG levels against J5 Bacterin® that exceeded their own baseline levels, and either exceeded (5 cows) or were similar (2 cows) to the anti-Bacterin IgG levels in the hyperimmunized cow (FIG. 8B).

### Example 13

### J5 LPS Vaccine Protects Against Shiga Toxin Toxicity

Endotoxemia is a critical component in the development of hemolytic uremic syndrome in a mouse model that closely mimics the condition in humans. Since this condition is caused by the ingestion of E. coli strains that express both a toxin (Shiga-like toxin 2) and LPS, oral administration of a colostrum enriched in antibodies to broadly cross-reactive epitopes in the LPS core region will treat and ameliorate the disease.

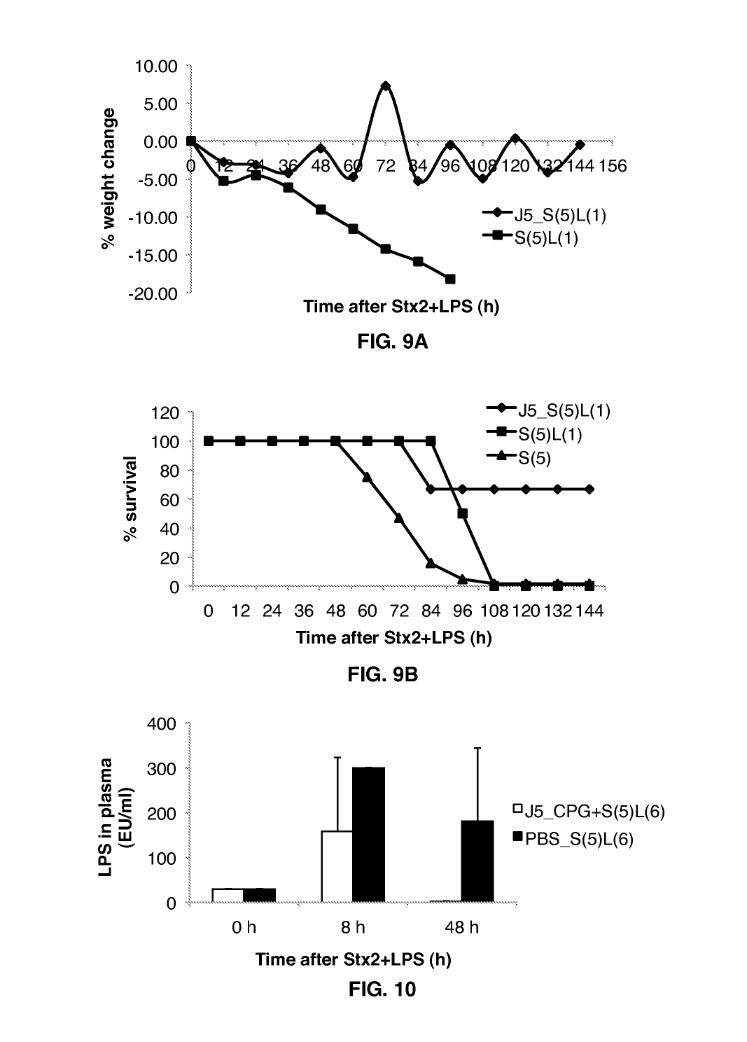

To examine the effect of J5dLPS/OMP immunization on mice given Shige-like toxin 2 and LPS, C57BL/6 male mice were immunized with the J5dLPS/OMP vaccine on days 0, 14 and 28. Control mice received an equal volume of PBS. At 14 days after the last dose of vaccine, mice were given Shiga toxin 2 (Stx2) at 5 ng/20 g body weight either alone (S(5), or with LPS 1 μg/20 g body weight. They were then followed for weight change (FIG. 9A) or survival (FIG. 9B). Mice that received the vaccine before Stx2/LPS challenge (J5_S(5)L(1)) did not lose weight while unimmunized mice lost >15% of their initial weight. Mice that were immunized with the vaccine before Stx2/LPS challenge (J5_S(5)L(1) had better survival than non-immunized mice that received either Stx2/LPS or Stx2 alone (right panel). J5dLPS/OMP immunization also reduced circulating endotoxin in mice administered Stx2LPS (FIG. 10).

Accordingly, in another aspect of the present invention, there is provided a method for treating an individual for hemolytic uremic syndrome or of protecting an individual against hemolytic uremic syndrome, comprising the step of administering to the patient an effective amount of a composition or formulation of colostrum enriched in anti-endotoxin antibodies. Generally, this composition is administered in a dose of from about 10 mg/ml to about 500 mg/ml.

### Example 14

### Bovine Colostrum Enriched in Broadly Cross-Reactive Anti-Endotoxin Antibodies Elicited by Immunization of Cows with J5/OMP Vaccine Protects Against Lethal Sepsis

A study of J5/OMP vaccine has been conducted in an animal model of sepsis. Lab rats were made neutropenic with cyclophosphamide. Three doses of antibiotic were given to wipe out their endogenous flora and the rats were challenged orally with a lethal clinical isolate of Pseudomonas aeruginosa. The rats were treated with 2 ml/day on days 3-8 with either PBS, non-immune bovine colostrum or hyperimmune bovine colostrum from cows immunized with the J5/OMP vaccine.

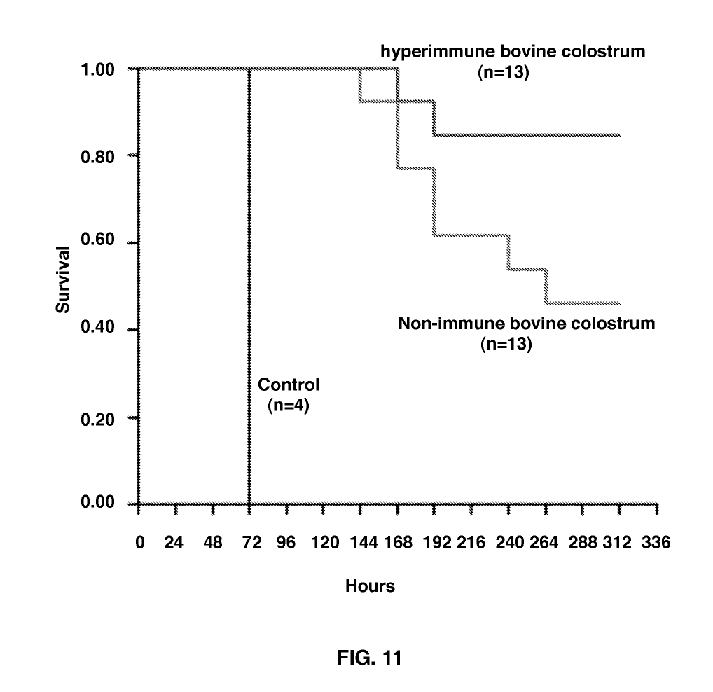

As shown in FIG. 11, animals receiving the PBS died quickly (n=4). Animals that received the non-immune bovine colostrum had ˜50% mortality (middle line), while the rats receiving the hyperimmune bovine colostrum (top line) had >90% survival. Further examination of these rats at the termination of the experiment demonstrated that there was a marked reduction in the bacterial colony counts in the lung and spleen of the animals receiving the hyperimmune bovine colostrum.

The following references are cited herein:
1. Liu et al., Infect Dis 2011, 204:282-290.
2. Hughes et al., J Immunol 2009, 183:2818-2826.
3. Bhattacharjee et al., J. Infect Dis 1996, 173:1157-1163.
4. Opal et al. Shock 2001, 15(4):285.
5. Opal et al., J. Infect. Dis 2005, 192:2074-2080.
6. Cross et al., Infect Immun 1993, 61:2741-2747.
7. Balan et al. Brain Res, 2010, 1316C:112-119.
8. Gray et al., Lancet Neurol, 2007, 6(5):397-406.
9. Mayo et al., J Immunol, 2008, 181(1):92-103.

What is claimed is:

1. A colostrum composition, comprising:
colostrum enriched in anti-endotoxin antibodies.

2. The composition of claim 1, wherein the level of said anti-endotoxin antibodies is from about 10 mg/ml to about 500 mg/ml.

3. The composition of claim 1, wherein said anti-endotoxin antibodies have high levels of cross reactivity with gram negative bacteria.

4. The composition of claim 3, wherein the gram negative bacteria are selected from the group consisting of Salmonella spp., Yersinia spp., Klebsiella spp., Shigella spp., Serratia spp., Helicobacter spp., Vibrio spp., and Pseudomonas spp.

5. The composition of claim 1, wherein said colostrum is selected from the group consisting of bovine colostrum, caprine colostrum, camelid colostrum, porcine colostrum and equine colostrum.

6. A pharmaceutical composition comprising the composition of claim 1.

7. The pharmaceutical composition of claim 6, wherein the composition is formulated as capsule, blister pack or sachet.

8. The composition of claim 1, further comprising glutamine or peptide analogs thereof.

9. A pharmaceutical composition comprising the composition of claim 8.

10. The pharmaceutical composition of claim 9, wherein the composition is formulated as capsule, blister pack or sachet.

11. A method for reducing endotoxemia in a patient or blocking the onset of a blood infection of a patient, comprising the step of:
administering to said patient an effective amount of the composition of claim 6.

12. The method of claim 11, wherein said composition is formulated as an oral dose formulation.

13. The method of claim 12, wherein said composition is formulated as capsule, blister pack or sachet.

14. The method of claim 12, wherein said composition is administered in a dose of from about 10 mg/ml to about 500 mg/ml.

15. A method for reducing endotoxemia in a patient or blocking the onset of a blood infection in a patient, comprising the step of:
administering to said patient an effective amount of the composition of claim 11.

16. The method of claim 15, wherein the composition is formulated as an oral dose formulation.

17. The method of claim 16, wherein the composition is formulated as capsule, blister pack or sachet.

18. The method of claim 15, wherein said composition is administered in a dose of from about 10 mg/ml to about 500 mg/ml.

19. A method for treating an individual for hemolytic uremic syndrome or of protecting an individual against hemolytic uremic syndrome, comprising the step of:
administering to said patient an effective amount of the composition of claim 6.

20. The method of claim 19, wherein said composition is administered in a dose of from about 10 mg/ml to about 500 mg/ml.

21. A method for treating an individual for hemolytic uremic syndrome or of protecting an individual against hemolytic uremic syndrome, comprising the step of:
administering to said patient an effective amount of the composition of claim 6.

22. The method of claim 21, wherein said composition is administered in a dose of from about 10 mg/ml to about 500 mg/ml.
